(19) United States
(12) Patent Application Publication
(10) Pub. No.: US 2009/0291488 A1
(43) Pub. Date: Nov. 26, 2009

(54) MICROORGANISM TESTING DEVICE AND MICROORGANISM TESTING CHIP

(75) Inventors: Kei TAKENAKA, Kashiwa (JP); Yasuhiko SASAKI, Tsuchiura (JP); Hideki NAKAMOTO, Tokai (JP); Kazuo TAKEI, Tokai (JP); Masahiro KURIHARA, Yokohama (JP); Yuusuke WATANABE, Hitachi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignee: Hitachi Engineering & Services Co., Ltd.

(21) Appl. No.: 12/468,885

(22) Filed: May 20, 2009

(30) Foreign Application Priority Data
May 20, 2008 (JP) 2008-131462

(51) Int. Cl. C12M 1/00

(52) U.S. Cl. 435/288.7

(57) ABSTRACT

A microorganism testing device includes: an analysis chip that includes a fluid specimen container, a reaction container for causing the fluid specimen to react with a reagent solution, a microorganism detection flow path, and an alignment reagent container for holding an alignment reagent to be used in an alignment work of the microorganism detection flow path; a moving stage for holding the analysis chip; and a detection device including a light source for irradiating the detection flow path with light, and an optical detector for detecting light from the detection flow path and converting the light into electric signals, in which the alignment reagent container is provided on a downstream side of the specimen container and the reaction container.

### BACKGROUND OF THE INVENTION

### Field of the Invention

The present invention relates to microorganism testing devices for measuring microorganisms, and microorganism testing chips.

Conventionally, there have been known measuring devices that execute various kinds of methods for quick and simple measurement of the number of viable bacteria. Especially, a device for measuring the number of bacteria, using a fluorescence flow cytometry method, is known as a technique for quick and direct measurement of the number of viable bacteria.

The fluorescence flow cytometry method is a particle measuring method, in which the diameter of specimen flow including specimens dyed with a fluorescent dye is made smaller, and the specimen is discharged in a flow one by one for measurement. A device for measuring the number of bacteria by using the above method is capable of measuring a specimen one by one in a short time.

Moreover, in the fluorescence flow cytometry method, in order to prevent elements in the specimen from adhering to the wall of the flow path, the diameter of the specimen flow is narrowed down by forming a laminar flow of a specimen and sheath liquid and making use of a pressure difference between those of two liquids.

Furthermore, in order to implement the method with a low cost, or to eliminate cleaning processing, there has been known a method in which a disposable chip is used as a flow path portion used for measurement by the fluorescence flow cytometry method, so that the flow path portion to be measured is disposed of without being re-used after measurement being made in the disposable chip. The method has been described in, for example, Journal of Biomolecular Techniques, Vol. 14, Issue 2, pp. 119-127.

### SUMMARY OF THE INVENTION

In the measuring device using the above-described fluorescence flow cytometry method, in order to irradiate the narrow detection flow path with excitation light and detect fluorescence emitted by a fine particle, it is necessary to align the position of the flow path with the focal points of the excitation light and the detector. However, the focus ranges of the excitation light and the detector and the width of the flow path are in the range of several microns to several hundreds of microns, so in order to perform accurate measurement, alignment with the accuracy of several tens of microns is required. Furthermore, in cases where the flow path portion is made into a disposable chip, the alignment of the flow path needs to be performed for each measurement.

As the means for aligning a chip, there has been known a method, in which a specific mark is placed on a part of the chip, and on the basis of the mark position, the positioning of the detection flow path is performed. However, since the chip is typically prepared by resin injection molding, the thermal hydraulic phenomenon of resin in the mold or the shrinkage deformation of the molded part after mold release occurs and thus the relative position between the detection flow path and the mark cannot be stabilized. For this reason, in order to reproduce the accuracy of several tens of microns, machining needs to be performed after injection molding but is unsuitable for the disposable chip. Moreover, other than an optical system for detecting fluorescence emitted from a fine particle, an optical system for recognizing the mark is required and as a result the size or cost of the device may be increased.

In order to solve the above-described problems, according to the present invention, a microorganism testing device includes: an analysis chip that includes at least a specimen container for holding a fluid specimen containing microorganisms, a reaction container for holding a reagent solution that reacts with the fluid specimen and reacting the fluid specimen with the reagent solution, a detection flow path for detecting the microorganisms, and an alignment reagent container that holds an alignment reagent for performing alignment of the detection flow path; a delivery device for delivering at least the fluid specimen, the reagent solution, and the alignment reagent to the analysis chip, the delivery device being connected to the analysis chip; a stage for holding the analysis chip and moving the analysis chip; a detection device that includes at least a light source for irradiating the detection flow path with light, and an optical detector for detecting light from the detection flow path and converting the light into electric signals; and an output device for outputting electric signals resulting from the conversion performed by the optical detector, in which the alignment reagent container is provided on a downstream side of at least the specimen container and the reaction container.

According to the present invention, high-precision alignment of a microorganism detection flow path can be achieved.

### BRIEF DESCRIPTION OF THE DRAWINGS

FIG. 1 is a configuration diagram of a microorganism testing device according to an embodiment of the present invention.

FIG. 2 is a schematic diagram illustrating the mechanism of positioning in the microorganism testing device of the present invention.

FIG. 3 is a schematic diagram illustrating the mechanism of positioning in the microorganism testing device of the present invention.

FIG. 4 is a schematic diagram of an image of a microorganism detection flow path in the microorganism testing device of the present invention.

FIG. 5 is a flow diagram of a procedure of alignment of an analysis chip 10 in the microorganism testing device of the present invention.

FIG. 6 is a plan view showing an analysis chip in a microorganism testing device according to an embodiment of the present invention.

FIG. 7 is a configuration diagram showing a detection device in a microorganism testing device according to an embodiment of the present invention.

FIG. 8 is a flow diagram of an analysis process in a microorganism testing device according to an embodiment of the present invention.

FIG. 9 is a plan view showing an analysis chip in a microorganism testing device according to an embodiment of the present invention.

FIG. 10 is a plan view showing an analysis chip in a microorganism testing device according to an embodiment of the present invention.

### DESCRIPTION OF THE PREFERRED EMBODIMENTS

Hereinafter, embodiments of the present invention will be described with reference to the accompanying drawings.

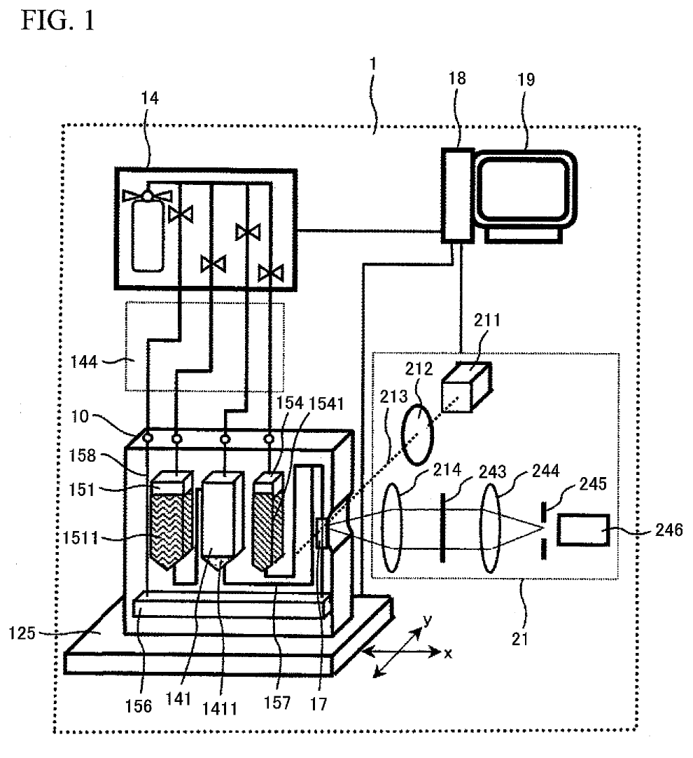

FIG. 1 is a configuration diagram of a microorganism testing device 1 of the present invention. The microorganism testing device 1 includes: an analysis chip 10 that holds a specimen and a reagent therein and is provided with a mechanism for performing processes necessary for measuring microorganisms; a delivery device 14 that controls the delivery of a specimen and a reagent in the analysis chip 10 through a chip connecting tube 144 connected to the analysis chip 10 in order to perform processes necessary for measuring microorganisms; an X-Y movable stage 125 for holding the analysis chip 10 and adjusting the position of the analysis chip 10; and a detection device 21 that irradiates microorganisms in the analysis chip 10 with excitation light and converts fluorescence from the microorganisms into electric signals. A system device 18 connected to the microorganism testing device 1 outputs control signals to the delivery device 14 and processes electric signals from the detection device 21. The measurement results obtained by processing of electric signals are displayed on an output device 19.

The analysis chip 10 includes: a specimen container 151 for holding a specimen 1511; a reaction container 141 that holds a dyeing reagent (reagent solution) 1411 for dyeing microorganisms in a specimen and that mixes the specimen and the dyeing reagent for reaction; an alignment reagent container 154 for holding a fluorescent alignment reagent 1541; a microorganism detection flow path 17 which is irradiated with excitation light 213 from an excitation light source 211 and which observes fluorescence of microorganisms or fluorescence of the alignment reagent; a detection liquid waste container 156 for discarding a liquid mixture of the specimen 1511 and the dyeing reagent 1411 that have passed through the microorganism detection flow path 17; a solution flow path 157 which connects the specimen container 151, the reaction container 141, and the microorganism detection flow path 17 and in which the specimen 1511 and the liquid mixture flow; and an air flow path 158 that connects the delivery device 14 to each of the above-described containers in order to flow the specimen 1511 and the liquid mixture by air pressure. The wavelength of excitation light and the wavelength of fluorescence of the dyeing reagent 1411 and the wavelength of excitation light and the wavelength of fluorescence of the alignment reagent 1541 are the same, or the difference between these wavelengths is within several tens of nm. Moreover, in order to prevent contamination to other reagents at the time of testing microorganisms, the alignment reagent container 154 is provided at a place (on the downstream side) nearer to the microorganism detection flow path 17 than other containers. Furthermore, the flow path from the alignment reagent container 154 to the microorganism detection flow path 17 is a flow path different from the flow path of other containers. Here, the specimen container 151 side is defined as the upstream side along the flow of the fluid specimen, and the microorganism detection flow path 17 side is defined as the downstream side. The flow path from the alignment reagent container 154 to the microorganism detection flow path 17 and the flow path from the specimen container 151 to the microorganism detection flow path 17 through the reaction container 141 are connected between the reaction container 141 and the microorganism detection flow path 17.

The detection device 21 includes: an irradiation unit comprising the excitation light source 211 and a condenser lens 212 for condensing light from the excitation light source 211; an objective lens 214 that condenses fluorescence from microorganisms passing through the microorganism detection flow path 17 or from the alignment reagent into parallel light; a band pass filter 243 through which the fluorescence from the microorganisms or the alignment reagent passes; a condenser lens 244 for focusing the parallel light; a pinhole 245 used as a spatial filter for cutting stray light; and a detection unit serving as an optical detector 246 for detecting light that has passed through the band pass filter 243. The irradiation unit and the detection unit are arranged so that the mutual focal points may overlap with each other, and at the time of measurement the microorganism detection flow path 17 is adjusted to the focal point.

The alignment reagent 1541 is flowed into the microorganism detection flow path 17, and the analysis chip 10 is moved by the X-Y movable stage 125 so that the fluorescence amount of the alignment reagent 1541 emitted from the microorganism detection flow path 17 may become the maximum, thereby performing alignment of the analysis chip 10.

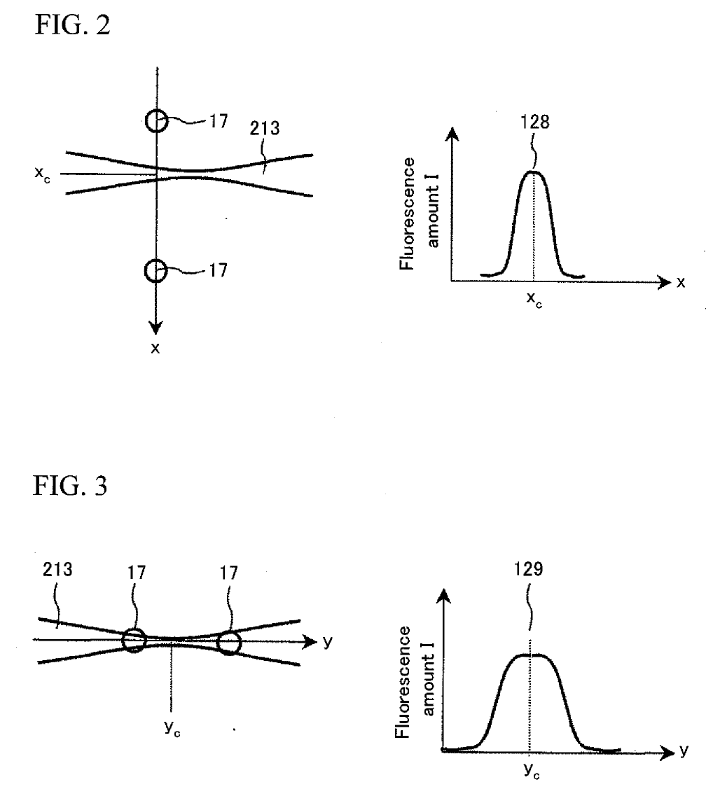

FIG. 2 and FIG. 3 are schematic diagrams illustrating the mechanism of positioning of the microorganism detection flow path 17.

After the analysis chip 10 is set to the X-Y movable stage 125, the alignment reagent 1541 is flowed into the microorganism detection flow path 17. Next, using the X-Y movable stage 125, the analysis chip 10 is moved in a direction (X direction) perpendicular to the optical axis of the excitation light 213 (FIG. 2). At this time, the relationship between a displacement in the X direction of the microorganism detection flow path 17 and a fluorescence amount I detected by the detection device 21 is as shown as a graph 128, and the fluorescence intensity becomes the highest when the microorganism detection flow path 17 passes through the optical axis of the excitation light 213 (at x_{c}). The profile of the displacement in the X direction and the fluorescence amount I detected by the detection device 21 is stocked in the system device 18. The position where the fluorescence amount I becomes the maximum or the primary differential of the fluorescence amount I with respect to the x direction becomes 0 is defined as x_{c}, to which the analysis chip 10 is moved. Next, the analysis chip is moved in a direction (Y direction) parallel to the optical axis of the excitation light 213 by the X-Y movable stage 125 (FIG. 3). At this time, the relationship between the displacement in the Y direction of the microorganism detection flow path and the fluorescence amount I detected by the detection device 21 is as shown as a graph 129, and the fluorescence intensity becomes the highest when the microorganism detection flow path 17 passes through the focal point of the excitation light 213 (at y_{c}). As with the x direction, the profile of the displacement in the Y direction and the fluorescence amount I detected by the detection device 21 is stocked in the system device 18. The position where the fluorescence amount I becomes the maximum or the primary differential of the fluorescence amount I with respect to the y direction becomes 0 is defined as y_{c}. Moreover, again, by repeating the operation of determining x_{c} and y_{c} with regard to the x direction and the y direction, the alignment can be performed more accurately.

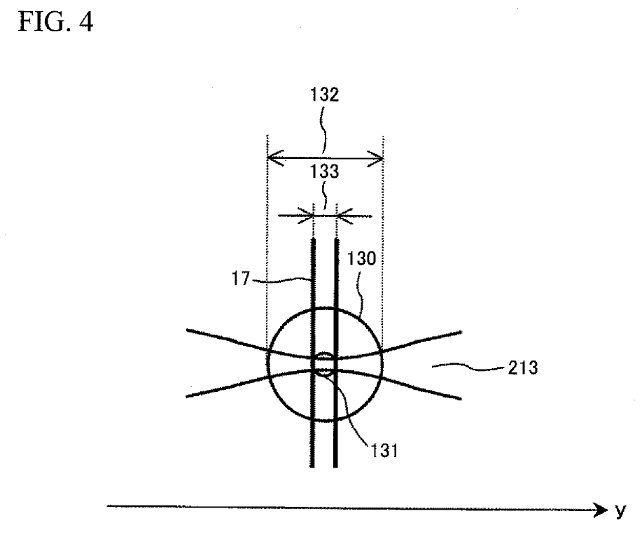

In performing the alignment in the Y direction, the alignment can be performed more accurately by using a pinhole having a smaller diameter located just before the optical detector. FIG. 4 is a schematic diagram of an image of the microorganism detection flow path 17 projected on the position of the pinhole. In the case of a pinhole 130, the fluorescence from the microorganism detection flow path 17 is detected when the position of the microorganism detection flow path 17 is within a range 132, while in the case of a smaller pinhole 131, the fluorescence from the microorganism detection flow path 17 is detected only when the position of the microorganism detection flow path 17 is within a range 133. Accordingly, the alignment can be performed more accurately.

If the diameter of the pinhole is too small, the intensity of light passing through the pinhole decreases. It is therefore preferable that the pinhole 131 having a small diameter be used in aligning the microorganism detection flow path 17, while in detecting microorganisms, the pinhole 130 having a large diameter be used in order to increase the light intensity to be detected.

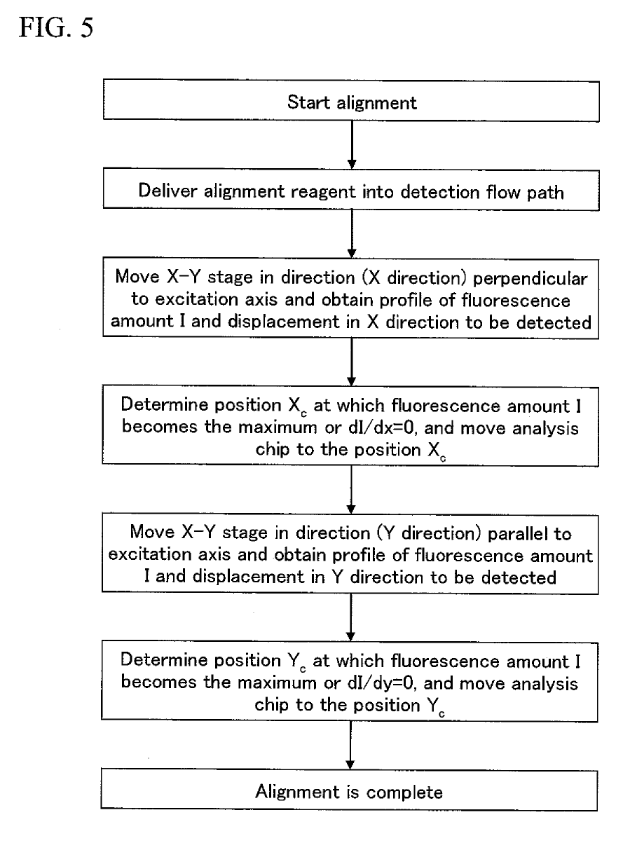

In FIG. 5, the above-described procedure for aligning the analysis chip 10 is depicted as a flow diagram.

Hereinafter, an embodiment for measuring the number of viable bacteria in a specimen taken from a food is described.

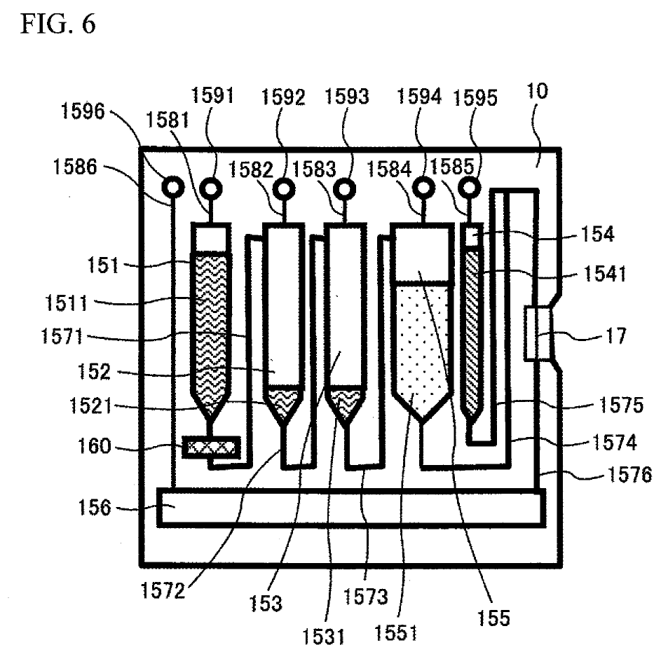

FIG. 6 is a plan view of the analysis chip 10 used in the microorganism testing device of the present invention. First, a configuration of the analysis chip 10 is described.

The analysis chip 10 includes: the specimen container 151 for holding the specimen 1511; a killed bacteria dyeing reagent container 152 for holding killed bacteria dyeing pigment 1521; a viable-and-killed bacteria dyeing reagent container 153 serving as the reaction container for holding viable-and-killed bacteria dyeing pigment 1531; the alignment reagent container 154 for holding the alignment reagent 1541; a cleaning liquid container 155 for holding cleaning liquid 1551; a food residue removing portion 160 serving as the filter for removing food residues contained in a specimen; the microorganism detection flow path 17 which is irradiated with excitation light from an external light source and which observes fluorescence of microorganisms; the detection liquid waste container 156 for discarding a liquid mixture of the specimen 1511, the killed bacteria dyeing reagent 1521, and the viable-and-killed bacteria dyeing pigment 1531 that have passed through the microorganism detection flow path 17; solution flow paths 1571-1576 which connect the specimen container 151, the food residue removing portion 160, the killed bacteria dyeing reagent container 152, the viable-and-killed bacteria dyeing reagent container 153, and the microorganism detection flow path 17 and through which the specimen 1511 and the liquid mixture flow; ventilation ports 1591-1596 through which the specimen 1511 and the liquid mixture in each of the above-described containers flow by the atmospheric pressure; and air flow paths 1581-1586 that connect the ventilation ports 1591-1596 to the respective containers. From the names of the containers to be connected, the solution flow paths 1571-1576, the ventilation ports 1591-1596, and the air flow paths 1581-1586 respectively are referred to as the flow path between specimen container-killed bacteria dyeing reagent container 1571, the flow path between killed bacteria dyeing reagent container-viable-and-killed bacteria dyeing reagent container 1572, the flow path between viable-and-killed bacteria dyeing reagent container-cleaning liquid container 1573, the flow path between cleaning liquid container-microorganism detection flow path 1574, the flow path between alignment reagent container-microorganism detection flow path 1575, and the flow path between microorganism detection flow path-detection liquid waste container 1576; the specimen container ventilation port 1591, the killed bacteria dyeing reagent container ventilation port 1592, the viable-and-killed bacteria dyeing reagent container ventilation port 1593, the cleaning liquid container ventilation port 1594, the alignment reagent container ventilation port 1595, and the detection liquid waste container ventilation port 1596; and the specimen container air flow path 1581, the killed bacteria dyeing reagent container air flow path 1582, the viable-and-killed bacteria dyeing reagent container air flow path 1583, the cleaning liquid container air flow path 1584, the alignment reagent container air flow path 1585, and the detection liquid waste container air flow path 1586.

The specimen container 1511, the food residue removing portion 160, the killed bacteria dyeing reagent container 152, the viable-and-killed bacteria dyeing reagent container 153, the cleaning liquid container 155, the microorganism detection flow path 17, and the detection liquid waste container 156 are connected in series by the solution flow paths 1571-1574, and 1576. The alignment reagent container air flow path 1585 branches from the cleaning liquid container air flow path 1584.

The depth and width of the respective solution flow paths 1571-1576 are formed in the range of 10 μm to 1 mm, the depth and width of the respective air flow paths 1581-1586 are formed in the range of 10 μm to 1 mm, and the cross-sectional area of the respective solution flow paths 1571-1576 is formed so as to be larger than that of the air respective flow paths 1581-1586.

The microorganism detection flow path 17 needs to be excellent in the optical characteristics, such as optical transparency, profile irregularity, and refractive index, in order to perform fluorescence measurement. A tube of quartz or an optical glass excellent in the optical characteristics is connected to the analysis chip 10 prepared by a separate process and is used as the microorganism detection flow path 17, or the microorganism detection flow path 17 is prepared integrally with the analysis chip 10 by use of a resin material, such as polymethacrylic acid methyl ester or polydimethylsiloxane, excellent in the optical characteristics. Although the cross-sectional shape of the microorganism detection flow path 17 is preferably rectangular, it may be circular. The larger the cross section size of the microorganism detection flow path 17 becomes, the smaller the pressure loss becomes. However, since the size of the microorganism detection flow path 17 is preferably smaller in order to flow a microorganism one by one, one side of the rectangular preferably ranges from 1 μm to 1 mm in length and the length of the flow path preferably ranges from 0.01 mm to 10 mm. The optical axis of the excitation light with which the microorganism detection flow path 17 is irradiated is perpendicular to the microorganism detection flow path 17.

The killed bacteria dyeing reagent 1521, the viable-and-killed bacteria dyeing reagent 1531, and the alignment reagent 1541 are encapsulated beforehand in the analysis chip 10. The specimen 1511 is injected into the specimen container 151 from the ventilation port 1591 before testing, and the cleaning liquid 1551 is injected into the cleaning liquid container 155 from the ventilation port 1594 before testing.

The volume of the specimen container 151 is larger than that of the specimen 1511. The volume of the killed bacteria dyeing reagent container 152 is larger than the total volume of the specimen 1511 and the killed bacteria dyeing reagent 1521. The volume of the viable-and-killed bacteria dyeing reagent container 153 is larger than the total volume of the specimen 1511, the killed bacteria dyeing reagent 1521, and the viable-and-killed bacteria dyeing reagent 1531. The volume of the cleaning liquid container 155 is larger than the total volume of the specimen 1511, the killed bacteria dyeing reagent 1521, the viable-and-killed bacteria dyeing reagent 1531, and the cleaning liquid 1551. Moreover, the highest point of the flow path between specimen container-killed bacteria dyeing reagent container 1571 is formed so as to be higher than the water level of the specimen 1511 in the specimen container 151. Similarly, the highest point of the flow path between killed bacteria dyeing reagent container-viable-and-killed bacteria dyeing reagent container 1572 is formed so as to be higher than the water level of the liquid mixture of the specimen 1511 and water level of the killed bacteria dyeing pigment 1521. Furthermore, the highest point of the flow path between viable-and-killed bacteria dyeing reagent container-cleaning liquid container 1573 is formed so as to be higher than the water level of the liquid mixture of the specimen 1511, the killed bacteria dyeing pigment 1521, and the viable-and-killed bacteria dyeing pigment 1531. Moreover, the highest point of the flow path between cleaning liquid container-microorganism detection flow path 1574 is formed so as to be higher than the water level of the liquid mixture of the specimen 1511, the water level of the killed bacteria dyeing pigment 1521, the viable-and-killed bacteria dyeing pigment 1531, and the cleaning liquid 1551. Moreover, the highest point of the flow path between alignment reagent container-microorganism detection flow path 1575 is formed so as to be higher than the water level of the alignment reagent 1541.

The specimen 1511 used here is obtained by stomaching of a food to be inspected after adding physiological salt solution with a mass ratio of ten relative to the food to be inspected. The cleaning liquid 1551 is primarily composed of physiological salt solution or pure water.

For example, PI (propidium iodide) (0.1 μg/ml to 1 mg/ml) is used as the killed bacteria dyeing reagent 1521, and for example, DAPI (4′,6-diamidine 2′-phenylindole) (1 μg/ml to 1 mg/ml), AO (acridine orange) (1 μg/ml to 1 mg/ml), EB (ethidium bromide) (1 μg/ml to 1 mg/ml), LDS 751 (0.1 μg/ml to 1 mg/ml), or the like is used as the viable-and-killed bacteria dyeing reagent 1541.

A solution containing fluorochromes, such as PI, DAPI, AO, EB, or LDS 751, or a fine particle that emits fluorescence of a specific wavelength is used as the alignment reagent 1541. The wavelength peak of the alignment reagent 1541 is preferably close to that of the killed bacteria dyeing reagent 1521 or the viable-and-killed bacteria dyeing reagent 1531 because the optical system for detection can be used in common.

In measurement of the number of viable bacteria using the analysis chip 10, the alignment of the analysis chip is performed first and then the number of viable bacteria is measured. The flow of each of the liquids in each of the steps of microorganism measurement is described.

The flow of each of the liquids in aligning the analysis chip 10 is described first. The analysis chip 10 is used upright with the lower side thereof downward in the view. In positioning the analysis chip 10, the alignment reagent 1541 (here, PI (the wavelength peak: 532 nm) is used) is flowed into the microorganism detection flow path 17. Pressure from the delivery device 14 is applied to the alignment reagent container 154 through the alignment reagent container ventilation port 1595 to increase the air pressure therein. At the same time, the detection liquid waste container 156 is opened to the atmosphere through the detection liquid waste container ventilation port 1596. The alignment reagent 1541 enters the detection liquid waste container 156 through the microorganism detection flow path 17 by the pressure difference. Since the microorganism detection flow path 17 is being irradiated with the excitation light from the detection device, the alignment reagent 1541 emits fluorescence when passing through the microorganism detection flow path 17. The position of the analysis chip 10 is adjusted so that the fluorescence detected by the detection device may become the maximum. Subsequently, a part of the cleaning liquid 1551 is flowed into the microorganism detection flow path 17 in order to clean the microorganism detection flow path 17 after positioning the analysis chip 10, because the alignment reagent 1541 may remain in the microorganism detection flow path 17.

Next, the flow of each of the liquids in the step of measuring the number of viable bacteria is described.

1. The specimen 1511 is flowed into the killed bacteria dyeing reagent container 152. Pressure from the delivery device 14 is applied to the specimen container 151 through the ventilation port 1591 to increase the air pressure therein. At the same time, the killed bacteria dyeing reagent container 152 is opened to the atmosphere through the killed bacteria dyeing reagent container ventilation port 1592. The specimen 1511 enters the killed bacteria dyeing reagent container 152 by the pressure difference and mixes with the killed bacteria dyeing reagent 1521. The killed bacteria in the specimen 1511 is dyed with the killed bacteria dyeing reagent 1521 (here, PI (peak wavelength: 532 nm) is used).

On the other hand, the viable bacteria in the specimen 1511 are not dyed. The water level of the liquid mixture of two liquids does not exceed the highest point of the flow path between killed bacteria dyeing reagent container-viable-and-killed bacteria dyeing reagent container 1572 that connects the killed bacteria dyeing reagent container 152 and the viable-and-killed bacteria dyeing reagent container 153. Furthermore, the air in the killed bacteria dyeing reagent container 152 is discharged to the outside through the killed bacteria dyeing reagent container ventilation port 1592. Since the air pressure of the killed bacteria dyeing reagent container 152 is equal to the atmospheric pressure, the liquid mixture of two liquids is not pushed out to the viable-and-killed bacteria dyeing reagent container 153 and the liquid mixture can be held in the killed bacteria dyeing reagent container 152 during time required for the reaction. Similarly, the viable-and-killed bacteria dyeing reagent 1531 (here, LDS 751 (peak wavelength: 710 nm) is used) is not pushed out to the cleaning liquid container 155, and is not flowed backward to the killed bacteria dyeing reagent container 152.

At this time, in order to further prevent the liquid mixture from flowing into the viable-and-killed bacteria dyeing reagent container 153, the respective air pressures of the viable-and-killed bacteria dyeing reagent container 153, the alignment reagent container 154, the cleaning liquid container 155, and the detection liquid waste container 156 may be increased to within a range lower than the air pressure of the specimen container 151 by applying the pressure from the delivery device through each of the ventilation ports 1593-1596.

The influence on dyeing by a change in temperature is preferably reduced by keeping the temperature of the analysis chip 10 constant during dyeing.

Moreover, when the specimen 1511 flows into the killed bacteria reagent container 152 through the residual food removing portion 160, the residual foods in the specimen 1511 are removed from the specimen 1511 by the residual food removing portion 160.

2. The liquid mixture of the specimen 1511 and the killed bacteria dyeing reagent 1521 is flowed into the viable-and-killed bacteria dyeing reagent container 153. The viable-and-killed bacteria dyeing reagent 1531 is added to the liquid mixture, and the killed bacteria and viable bacteria in the specimen 1511 are dyed with the killed bacteria dyeing reagent 1531.

3. The liquid mixture of the specimen 1511, the killed bacteria dyeing reagent 1521, and the viable-and-killed bacteria dyeing reagent 1531 is flowed into the cleaning liquid container 155. The cleaning liquid 1541 is added to the liquid mixture, and the concentration of uncombined dye (the killed bacteria dyeing reagent 1521 and the viable-and-killed bacteria dyeing reagent 1531 that do not dye microorganisms) contained in the liquid mixture is reduced. Reduction of the concentration of uncombined dye reduces the fluorescence amount emitted by the uncombined dye that causes noise at the time of detection.

4. The liquid mixture of the specimen 1511, the killed bacteria dyeing reagent 1521, the viable-and-killed bacteria dyeing reagent 1531, and the cleaning liquid 1541 is flowed into the microorganism detection flow path 17. Since the excitation light is being applied from the direction perpendicular to the sheet of the drawing, the microorganisms emit fluorescence. As only the fluorescence of the viable-and-killed bacteria dyeing reagent 1531 is detected for viable bacteria, and the fluorescence of the viable-and-killed bacteria dyeing reagent 1531 and the fluorescence of the killed bacteria dyeing reagent 1521 are detected for killed bacteria in the detection device 21, it becomes possible to distinguish between the viable bacteria and the killed bacteria.

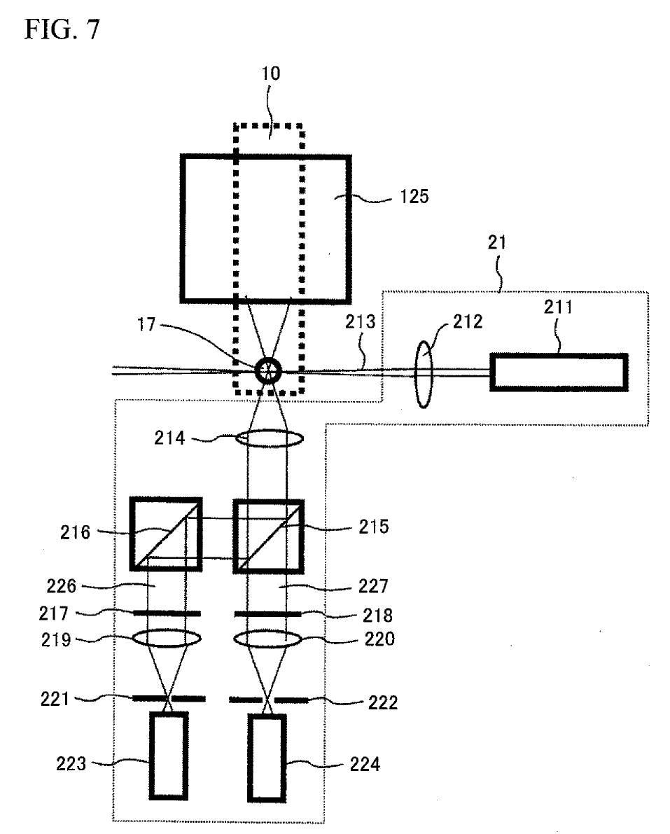

FIG. 7 is a configuration diagram of an optical system of the detection device 21 for distinguishing between viable bacteria and killed bacteria. The optical device and its arrangement may differ depending on both excitation spectra and fluorescence spectra of fluorochromes used. Here, an optical system suitable for use of two kinds of fluorochromes, i.e., PI as the killed bacteria dyeing reagent (the excited wavelength peak: 532 nm, and the fluorescence wavelength peak: 615 nm) and LDS 751 (the excited wavelength peak: 541 nm, and the fluorescence wavelength peak: 710 nm) as the viable-and-killed bacteria dyeing reagent, is described.

The detection device 21 includes: an irradiation system including the excitation light source 211 (the wavelength: 532 nm) and the condenser lens 212 for condensing light from the excitation light source 211; and a detection system including: the objective lens 214 that condenses fluorescence from microorganisms passing through the microorganism detection flow path 17 into parallel light; a dichroic mirror 215 for reflecting light with a wavelength of 610 nm or less and allowing light with a wavelength of 610 nm or more to pass therethrough; a mirror 216; a short wavelength band pass filter 217 for allowing only light with wavelengths near 610 nm to pass therethrough; a long wavelength band pass filter 218 for allowing light with wavelengths near 710 nm to pass therethrough; the condenser lens 219, 220 for focusing parallel light; the pinholes 221, 222 used as the spatial filter for cutting stray light; a short wavelength optical detector 223 for detecting light that has passed through the short wavelength band pass filter 217; and a long wavelength optical detector 224 for detecting light that has passed through the long wavelength band pass filter 218.

The condenser lens 212 and the objective lens 214 are constructed so that the focal points thereof may intersect with each other. At the time of testing, the microorganism detection flow path 17 of the analysis chip 10 is adjusted to the focal point of the condenser lens 212 and the objective lens 214 by the X-Y movable stage 125. The excitation light source 211 uses a laser, and the short wavelength light detector 223 and the long wavelength optical detector 224 use a photomultiplier, respectively.

The excitation light (the wavelength: 532 nm) output from the excitation light source 211 is condensed by the condenser lens 212 to excite PI and LDS 751 that have dyed the microorganisms flowing in the microorganism detection flow path 17. Fluorescence 226 from PI (the center wavelength: 610 nm) and fluorescence 227 from LDS 751 (the center wavelength: 710 nm) enter the condenser lens 220. Since the fluorescence 226 from PI is reflected by the dichroic mirror 215 and the fluorescence 227 from LDS 751 passes through the dichroic mirror 215, the fluorescence emitted from the two dyes can be separated based on the difference in the wavelengths. The fluorescence 226 from PI passes through the short wavelength band pass filter 217, is condensed by the condenser lens 219, passes through the pinhole 221, and enters the short wavelength optical detector 223, while the fluorescence 227 from LDS 751 passes through the long wavelength band pass filter 218, is condensed by the condenser lens 220, passes through the pinhole 222, and enters the long wavelength optical detector 224.

The incident fluorescence 226 is converted into electric signals by the short wavelength optical detector 223 and the incident fluorescence 227 is converted into electric signals by the long wavelength optical detector 224, and these electric signals are sent to the system device 18. The system device 18 processes the electric signals sent from the short wavelength optical detector 223 and the long wavelength optical detector 224, and then outputs information on the number of microorganisms to the output device 19 as the test result.

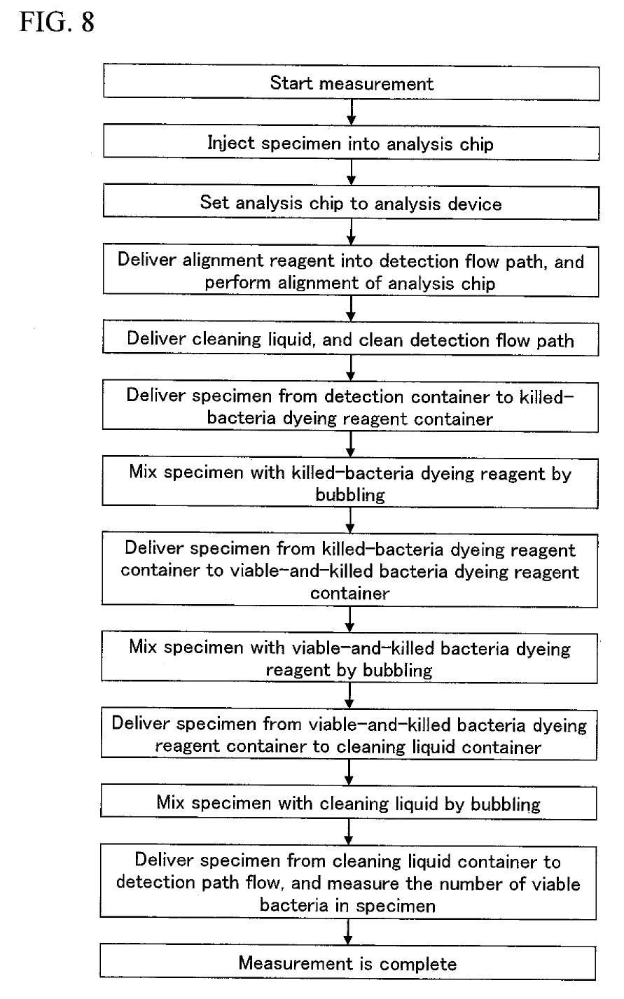

In FIG. 8, the above-described process to measure the number of viable bacteria is depicted as a flow diagram.

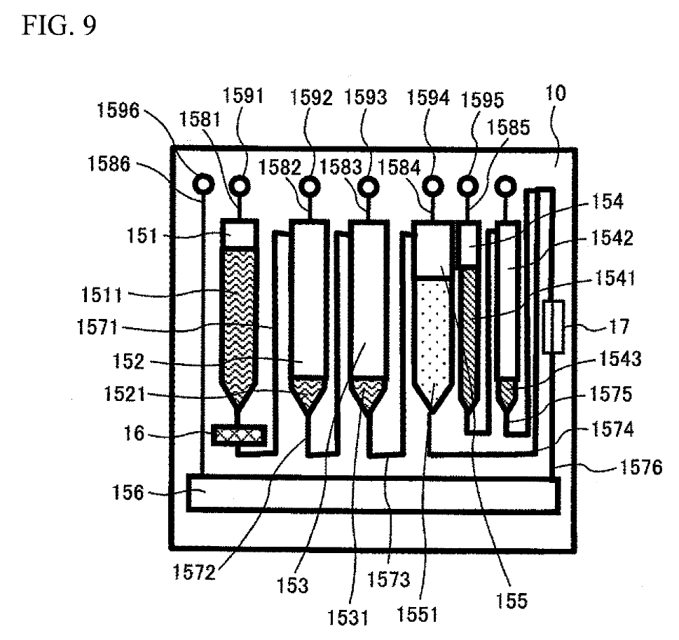

FIG. 9 is a plan view of the analysis chip 10 when a spontaneous-luminescence reagent is used as the alignment reagent. Containers for holding two reagents that are mixed to react and emit light are provided in the analysis chip 10. These containers are a first luminescence reagent container 154 for holding a first luminescence reagent 1541 and a second luminescence reagent container 1542 for holding a second luminescence reagent 1543. For example, with the use of luciferin-luciferase compound liquid as the first luminescence reagent 1541 and ATP aqueous solution as the second luminescence reagent 1543, the first luminescence reagent 1541 and the second luminescence reagent 1543 are mixed together, and thereafter the resultant liquid mixture is flowed into the microorganism detection flow path 17. Thereby, the positioning of the analysis chip 10 can be performed making use of the luminescence of ATP.

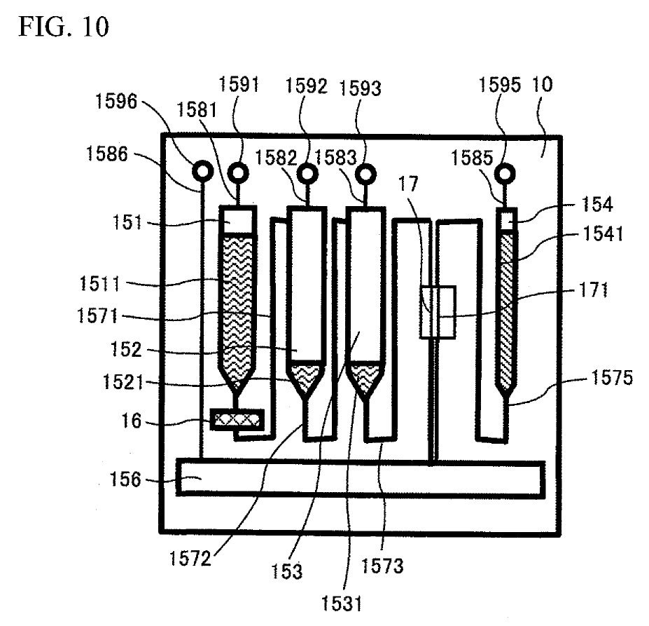

FIG. 10 is a plan view of the analysis chip 10 in which the flow path, where the alignment reagent flows, and the microorganism detection flow path 17 are separate flow paths. The alignment reagent 1541 is flowed into the alignment flow path 171 adjacent to the microorganism detection flow path 17 to perform the alignment of the analysis chip 10.

### EXPLANATION OF REFERENCE NUMERALS

1 . . . microorganism testing device
10 . . . analysis chip
14 . . . delivery device
17 . . . microorganism detection flow path
18 . . . system device
19 . . . output device
21 . . . detection device
125 . . . X-Y movable stage
128 . . . graph showing relationship between displacement in x direction and output value
129 . . . graph showing relationship between displacement in y direction and output value
130, 221, 222 . . . pinhole
131 . . . small pinhole
132, 133 . . . range
151 . . . specimen container
152 . . . killed bacteria dyeing reagent container
153 . . . viable-and-killed bacteria dyeing reagent container
154 . . . alignment reagent container
155 . . . cleaning liquid container
156 . . . detection liquid waste container
157 . . . solution flow path
158 . . . air flow path
159 . . . ventilation port
211 . . . excitation light source
212, 219, 220 . . . condenser lens
213 . . . excitation light
214 . . . objective lens
215 . . . dichroic mirror
216 . . . mirror
217 . . . short wavelength band pass filter
218 . . . long wavelength band pass filter
223 . . . short wavelength optical detector
224 . . . long wavelength optical detector
226 . . . fluorescence from PI
227 . . . fluorescence from LDS 751

What is claimed is:

1. A microorganism testing device comprising:
an analysis chip that includes at least
a specimen container for holding a fluid specimen containing microorganisms,
a reaction container for holding a reagent solution that reacts with the fluid specimen and for causing the fluid specimen to react with the reagent solution,
a detection flow path for detecting the microorganism, and
an alignment reagent container for holding an alignment reagent for performing alignment of the detection flow path;
a delivery device for delivering at least the fluid specimen, the reagent solution, and the alignment reagent in the analysis chip, the delivery device being connected to the analysis chip;
a stage for holding the analysis chip and moving the analysis chip;
a detection device including at least
a light source for irradiating the detection flow path with light, and
an optical detector for detecting light from the detection flow path and converting the light into electric signals; and
an output device for outputting electric signals resulting from the conversion performed by the optical detector, wherein
the alignment reagent container is provided on a downstream side of at least the specimen container and the reaction container.

2. The microorganism testing device according to claim 1, wherein a flow path from the alignment reagent container to the detection flow path is a flow path different from a flow path connecting the specimen container, the reaction container, and the detection flow path, and is connected between the reaction container and the detection flow path.

3. The microorganism testing device according to claim 2, wherein the highest point of the flow path from the alignment reagent container to the detection flow path is provided at a position higher than the highest point of the alignment reagent held in the alignment reagent container.

4. The microorganism testing device according to claim 1, wherein the analysis chip further includes a cleaning liquid container for holding a cleaning liquid for cleaning at least the detection flow path, the cleaning liquid container provided between the reaction container and the detection flow path.

5. A microorganism testing chip comprising:
a specimen container for holding a fluid specimen containing microorganisms;
a reaction container for holding a reagent solution that reacts with the fluid specimen and for causing the fluid specimen to react with the reagent solution;
a detection flow path for detecting the microorganism; and
an alignment reagent container for holding an alignment reagent for performing alignment of the detection flow path, wherein
the alignment reagent container is provided on a downstream side of at least the specimen container and the reaction container.

6. The microorganism testing chip according to claim 5, wherein a flow path from the alignment reagent container to the detection flow path is a flow path different from a flow path connecting the specimen container, the reaction container, and the detection flow path, and is connected between the reaction container and the detection flow path.

7. The microorganism testing chip according to claim 6, wherein the highest point of the flow path from the alignment reagent container to the detection flow path is provided at a position higher than the highest point of the alignment reagent held in the alignment reagent container.

8. The microorganism testing chip according to claim 5, further comprising a cleaning liquid container for holding a cleaning liquid for cleaning at least the detection flow path, the cleaning liquid container provided between the reaction container and the detection flow path.

9. A detecting device for a microorganism contained in a fluid specimen comprising:
an analysis chip including
a fluid specimen container,
a reaction container connected to the fluid specimen container via a first flow path and holding a reagent solution which reacts with the fluid specimen,
a microorganism detection flow path connected to the reaction container via a second flow path, and
an alignment reagent container connected to the microorganism detection flow path at the downstream side of the reaction container and holding an alignment reagent to be used in an alignment work of the microorganism detection flow path;
a delivery device connected to the fluid specimen container, the reaction container and the alignment reagent container via a connecting tube respectively and giving each container pressurized gas thereby the fluid specimen, the reagent solution or the alignment reagent is delivered to the next container or the microorganism detection flow path in the analysis chip;
a moving stage on which the analysis chip is hold;
a detection device including
a light source for irradiating the microorganism detection flow path with light, and
an optical detector for detecting light from the microorganism detection flow path and converting the light into electric signals; and
an output device for outputting electric signals resulting from the conversion performed by the optical detector.
